SEQUENCE LISTING (6 sequences)

SEQ ID NO: 1 - DNA, 7989 bp, Artificial Sequence
  HCV Replicon
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 2 - protein, 1985 aa, Artificial Sequence
  HCV Replicon
MAPITAYSQQTRGLLGCIITSLTGRDRNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGIDPNIRTGVRTITTGAPITYSTYGKFLADGGCSGGAYDIIICDECHSTDSTTILGIGTVLDQAETAGARLVVLATATPPGSVTVPHPNIEEVALSSTGEIPFYGKAIPIETIKGGRHLIFCHSKKKCDELAAKLSGLGLNAVAYYRGLDVSVIPTSGDVIVVATDALMTGFTGDFDSVIDCNTCVTQTVDFSLDPTFTIETTTVPQDAVSRSQRRGRTGRGRMGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTTTHPITKYIMACMSADLEVVTSTWVLVGGVLAALAAYCLTTGSVVIVGRIILSGKPAIIPDREVLYREFDEMEECASHLPYIEQGMQLAEQFKQKAIGLLQTATKQAEAAAPVVESKWRTLEAFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFFSCQRGYKGVWRGDGIMQTTCPCGAQITGHVKNGSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREEVTFLVGLNQYLVGSQLPCEPEPDVAVLTSMLTDPSHITAETAKRRLARGSPPSLASSSASQLSAPSLKATCTTRHDSPDADLIEANLLWRQEMGGNITRVESENKVVILDSFEPLQAEEDEREVSVPAEILRRSRKFPRAMPIWARPDYNPPLLESWKDPDYVPPVVHGCPLPPAKAPPIPPPRRKRTVVLSESTVSSALAELATKTFGSSESSAVDSGTATASPDQPSDDGDAGSDVESYSSMPPLEGEPGDPDLSDGSWSTVSEEASEDVVCCSMSYTWTGALITPCAAEETKLPINALSNSLLRHHNLVYATTSRSASLRQKKVTFDRLQVLDDHYRDVLKEMKAKASTVKAKLLSVEEACKLTPPHSARSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKAKKCPMGFAYDTRCFDSTVTENDIRVEESIYQCCDLAPEARQAIRSLTERLYIGGPLTNSKGQNCGYRRCRASGVLTTSCGNTLTCYLKAAAACRAAKLQDCTMLVCGDDLVVICESAGTQEDEASLRAFTEAMTRYSAPPGDPPKPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRARLLSQGGRAATCGKYLFNWAVRTKLKLTPIPAASQLDLSSWFVAGYSGGDIYHSLSRARPRWFMWCLLLLSVGVGIYLLPNR

SEQ ID NO: 3 - DNA, 21 bp, Artificial Sequence
  Primer
gggagagccatagtggtctgc

SEQ ID NO: 4 - DNA, 20 bp, Artificial Sequence
  Primer
cccaaatctccaggcattga

SEQ ID NO: 5 - DNA, 21 bp, Artificial Sequence
  Probe
cggaattgccaggacgaccgg

SEQ ID NO: 6 - DNA, 14002 bp, Artificial Sequence
  BVDV Replicon
gtatacgagaattagaaaaggcactcgtatacgtattgggcaattaaaaataataattaggcctagggaacaaatccctctcagcgaaagccgaaaagaggctagccatgcccttagtaggactagcataatgaggggggtagcaacagtggtgagttcgttggatggcttaagccctgagtacagggtagtcgtcagtggttcgacgccttggaataaaggtctcgagatgccacgtggacgagggcatgcccaaagcacatcttaacctgagcgggggtcgcccaggtaaaagcagttttaaccgactgttacgaatacagcctgatagggtgctgcagaggcccactgtattgctactaaaaatctctgctgtacatggcacatggagttgatcacaaatgaacttttatacaaaacatacaaacaaaaacccgtcggggtggaggaacctgtttatgatcaggcaggtgatcccttatttggtgaaaggggagcagtccaccctcaatcgacgctaaagctcccacacaagagaggggaacgcgatgttccaaccaacttggcatccttaccaaaaagaggtgactgcaggtcgggtaatagcagaggacctgtgagcgggatctacctgaagccagggccactattttaccaggactataaaggtcccgtctatcacagggccccgctggagctctttgaggagggatccgagcgcgctgaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattattccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggtgcgcgctagaatgcagatcttcgtgaagaccctgacgggcaagaccatcactcttgaggtcgagcccagtgacaccatcgagaatgtcaaggccaagatccaagacaaggaaggcatcccacctgaccagcagaggctgatattcgcgggcaaacagctggaggatggccgcaccctgtccgactacaacatccagaaagagtccaccttgcacctggtgctgcgtctccgcggtggcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatcgatagggagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaacgtctaggccccccgaaccacggggacgtggttttcctttgaaaaacacgataataccatggggcctgccgtgtgtaagaagatcacagagcacgaaaaatgccacattaatatactggataaactaaccgcatttttcgggatcatgccaagggggactacacccagagccccggtgaggttccctacgagcttactaaaagtgaggaggggtctggagactggctgggcttacacacaccaaggcgggataagttcagtcgaccatgtaaccgccggaaaagatctactggtctgtgacagcatgggacgaactagagtggtttgccaaagcaacaacaggttgaccgatgagacagagtatggcgtcaagactgactcagggtgcccagacggtgccagatgttatgtgttaaatccagaggccgttaacatatcaggatccaaaggggcagtcgttcacctccaaaagacaggtggagaattcacgtgtgtcaccgcatcaggcacaccggctttcttcgacctaaaaaacttgaaaggatggtcaggcttgcctatatttgaagcctccagcgggagggtggttggcagagtcaaagtagggaagaatgaagagtctaaacctacaaaaataatgagtggaatccagaccgtctcaaaaaacacagcagacctgaccgagatggtcaagaagataaccagcatgaacaggggagacttcaagcagattactttggcaacaggggcaggcaaaaccacagaactcccaaaagcagttatagaggagataggaagacacaagagagtattagttcttataccattaagggcagcggcagagtcagtctaccagtatatgagattgaaacacccaagcatctcttttaacctaaggataggggacatgaaagagggggacatggcaaccgggataacctatgcatcatacgggtacttctgccaaatgcctcaaccaaagctcagagctgctatggtagaatactcatacatattcttagatgaataccattgtgccactcctgaacaactggcaattatcgggaagatccacagattttcagagagtataagggttgtcgccatgactgccacgccagcagggtcggtgaccacaacaggtcaaaagcacccaatagaggaattcatagcccccgaggtaatgaaaggggaggatcttggtagtcagttccttgatatagcagggttaaaaataccagtggatgagatgaaaggcaatatgttggtttttgtaccaacgagaaacatggcagtagaggtagcaaagaagctaaaagctaagggctataactctggatactattacagtggagaggatccagccaatctgagagttgtgacatcacaatccccctatgtaatcgtggctacaaatgctattgaatcaggagtgacactaccagatttggacacggttatagacacggggttgaaatgtgaaaagagggtgagggtatcatcaaagatacccttcatcgtaacaggccttaagaggatggccgtgactgtgggtgagcaggcgcagcgtaggggcagagtaggtagagtgaaacccgggaggtattataggagccaggaaacagcaacagggtcaaaggactaccactatgacctcttgcaggcacaaagatacgggattgaggatggaatcaacgtgacgaaatcctttagggagatgaattacgattggagcctatacgaggaggacagcctactaataacccagctggaaatactaaataatctactcatctcagaagacttgccagccgctgttaagaacataatggccaggactgatcacccagagccaatccaacttgcatacaacagctatgaagtccaggtcccggtcctgttcccaaaaataaggaatggagaagtcacagacacctacgaaaattactcgtttctaaatgccagaaagttaggggaggatgtgcccgtgtatatctacgctactgaagatgaggatctggcagttgaccttttagggctagatggcctgatcctgggaaccagcaggtagtggagactggtaaagcactgaagcaagtgaccgggttgtcctcggctgaaaatgccctactagtggctttatttgggtatgtgggttaccaggctctctcaaagaggcatgtcccaatgataacagacatatataccatcgaggaccagagactagaagacaccacccacctccagtatgcacccaacgccataaaaaccgatgggacagagactgaactgaaagaactggcgtcgggtgacgtggaaaaaatcatgggagccatttcagattatgcagctgggggactggagtttgttaaatcccaagcagaaaagataaaaacagctcctttgtttaaagaaaacgtagaagctgcaaaagggtatgtccaaaaattcattgactcattaattgaaaataaagaagaaataatcagatatggtttgtggggaacacacacagcactatacaaaagcatagctgcaagactggggcatgaaacagcgtttgccacactagtgttaaagtggctagcttttggaggggaatcagtgtcagaccacgtcaagcaggcggcagttgatttagtggtctattatgtgatgaataagccttccttcccaggtgactccgagacacagcaagaagggaggcgattcgtcgcaagcctgttcatctccgcactggcaacctacacatacaaaacttggaattaccacaatctctctaaagtggtggaaccagccctggcttacctcccctatgctaccagcgcattaaaaatgttcaccccaacgcggctggagagcatggtgatactgagcaccacgatatataaaacatacctctctataaggaaggggaagagtgatggattgctgggtacggggataagtgcagccatggaaatcctgtcacaaaacccagtatcggtaggtatatctgtgatgttgggggtaggggcaatcgctgcgcacaacgctattgagtccagtgaacagaaaaggaccctacttatgaaggtgtttgtaaagaacttcttggatcaggctgcaacagatgagctggtaaaagaaaacccagaaaaaattataatggccttatttgaagcagtccagacaattggtaaccccctgagactaatataccacctgtatggggtttactacaaaggttgggaggccaaggaactatctgagaggacagcaggcagaaacttattcacattgataatgtttgaagccttcgagttattagggatggactcacaagggaaaataaggaacctgtccggaaattacattttggatttgatatacggcctacacaagcaaatcaacagagggctgaagaaaatggtactggggtgggcccctgcaccctttagttgtgactggacccctagtgacgagaggatcagattgccaacagacaactatttgagggtagaaaccaggtgcccatgtggctatgagatgaaagctttcaaaaatgtaggtggcaaacttaccaaagtggaggagagcgggcctttcctatgtagaaacagacctggtaggggaccagtcaactacagagtcaccaagtactacgatgacaacctcagagagataaaaccagtagcaaagttggaaggacaggtagagcactactacaaaggggtcacagcaaaaattgactacagtaaaggaaaaatgctcttggccactgacaagtgggaggtggaacatggtgtcataaccaggttagctaagagatatactggggtcgggttcaatggtgcatacttaggtgacgagcccaatcaccgtgctctagtggagagggactgtgcaactataaccaaaaacacagtacagtttctaaaaatgaagaaggggtgtgcgttcacctatgacctgaccatctccaatctgaccaggctcatcgaactagtacacaggaacaatcttgaagagaaggaaatacccaccgctacggtcaccacatggctagcttacaccttcgtgaatgaagacgtagggactataaaaccagtctaggagagagagtaatccccgaccctgtagttgatatcaatttacaaccagaggtgcaagtggacacgtcagaggttggggtcacaataattggaagggaaaccctgatgacaacgggagtgacacctgtcttggaaaaagtagagcctgacgccagcgacaaccaaaacttggtgaagatcgggttggatgagggtaattacccagggcctggaatacggacacatacactaacagaagaaatacacaacagggatgcgaggcccttcatcatgatcctgggctcaaggaattccatatcaaatagggcaaagactgctagaaatataaatctgtacacaggaaatgaccccagggaaatacgagacttgatggctgcagggcgcatgttagtagtagcactgagggatgtcgaccctgagctgtctgaaatggtcgatttcaaggggacttttttagatagggaggccctggaggctctaagtctcgggcaacctaagccgaagcaggttaccaaggaagctgttaggaatttgatagaacagaaaaaagatgtggagatccctaactggtttgcatcagatgacccagtatttctggaagtggccttaaaaaatgataagtactacttagtaggagatgttggagaggtaaaagatcaagctaaagcacttggggccacggatcagacaagaattataaaggaggtaggctcaaggacgtatgccatgaagctatctagctggttcctccaggcatcaaacaaacagatgagtttaactccactgtttgaggaattgttgctacggtgcccacctgcaactaagagcaataaggggcacatggcatcagcttaccaattggcacagggtaactgggagcccctcggttgcggggtgcacctaggtacaataccagccagaagggtgaagatacacccatatgaagcttacctgaagttgaaagatttcatagaagaagaagagaagaaacctagggttaaggatacagtaataagagagcacaacaaatggatacttaaaaaaataaggtttcaaggaaacctcaacaccaagaaaatgctcaaccctgggaaactatctgaacagttggacagggaggggcgcaagaggaacatctacaaccaccagattggtactataatgtcaagtgcaggcataaggctggagaaattgccaatagtgagggcccaaaccgacaccaaaacctttcatgaggcaataagagataagatagacaagagagaaaaccggcaaaatccagaattgcacaacaaattgttggagattttccacacgatagcccaacccgccctgaaacacacctacggtgaggtgacgtgggagcaacttgaggcggggataaatagaaagggggcagcaggcttcctggagaagaagaacatcggagaagtattggattcagaaaagcacctggtagaacaattggtcagggatctgaaggccgggagaaagataaaatattatgaaactgcaataccaaaaaatgagaagagagatgtcagtgatgactggcaggcaggggacctggtggatgagaagaggccaagagttatccaataccctgaagccaagacaaggctagccatcactaaggtcatgtataactgggtgaaacagcagcccgttgtgattccaggatatgaaggaaagacccccttgttcaacatctttgataaggtgagaaaggaatgggacttgttcaatgagccagtggccgtaagttttgacaccaaagcctgggacactcaagtgactagtaaggatctgcaacttattggagaaatccagaaatattactataagaaggagtggcacaagttcattgacaccatcaccgaccacatgacagaagtaccagttataacagcagatggtgaagtatatataagaaatgggcagagagggagcggccagccagacacaagtgctggcaacagcatgttaaatgtcctgacaatgatgtacgccttctgcgaaagcacaggggtaccgtacaagagtttaacagggtggcaaggatccacgtctgtggggatgatggcttcttaataactgaaaaagggttagggctgaaatttgctaacaaagggatgcagattcttcatgaagcaggcaaacctcagaagataacggaaggggaaaagatgaaagttgcctatagatttgaggatatagagttctgttctcataccccagtccctgttaggtggtccgacaacaccagtagtcacatggccgggagagacaccgctgtgatactatcaaagatggcaacaagattggattcaagtggagagatgggtaccacagcatatgaaaaagcggtagccttcagtttcttgctgatgtattcctggaacccgcttgttaggaggatttgcctgttggtcctttcgcaacagccagagacagacccatcaaaacatgccacttattattacaaaggtgatccaataggggcctataaagatgtgataggtcggaatctaagtgaactgaagagaacaggctttgagaaattggcaaatctaaacctaagcctgtccacgttggggatctggactaagcacacaagcaaaagaataattcaggactgtgttgccattgggaaagaagagggcaactggctagttaacgccgacaggctgatatccagcaaaactggccacttatacatacctgataaaggctttacattacaaggaaagcattatgagcaactgcagctaagaacagagacaaacccggtcatgggggttgggactgagagatacaagttaggtcccatagtcaatctgctgctgaggaggttgaaaattctgctcatggcggccgtcggcgtcagcagctgagacaaaatgtatatattgtaaataaattaatccatgtacatagtgtatataaatatagttgggaccgtccacctcaagaagacgacacgcccaacacgcacagctaaacagtagttaagattatctacctcaagataacactacatttaatgcacacagcactttagctgtatgaggatacgcccgacgtctatagttggactagggaagacctctaacagccccctctagactgccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacaaaatattaacgtttacaatttcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacttggtca

(19) United States
(12) Patent Application Publication
(10) Pub. No.: US 2005/0260568 A1
(43) Pub. Date: Nov. 24, 2005

(54) Hepatitis C virus assays

(76) Inventors: Min Gao, Madison, CT (US); Julie A. Lemm, Durham, CT (US); Donald R. O'Boyle, Clinton, CT (US); Peter Nower, Wethersfield, CT (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 11/119,330

(22) Filed: Apr. 29, 2005

Related U.S. Application Data
(60) Provisional application No. 60/567,270, filed on Apr. 30, 2004.
(60) Provisional application No. 60/568,590, filed on May 6, 2004.

(51) Int. Cl. C12Q 1/70; C12Q 1/68

(52) U.S. Cl. 435/5; 435/6

(57) ABSTRACT

The present invention includes assays useful for identifying inhibitors of Hepatitis C virus (HCV) activity. Particularly, the present invention is directed to a HCV assay useful for high throughput screening that quantifies both the amount of HCV RNA replication inhibitory activity associated with a test compound and the amount of cytotoxicity associated with that test compound, as well as the specificity of that compound for HCV over closely-related viruses (such as Bovine viral diarrhea virus). The present invention also includes compounds discovered using this assay, compositions containing such compounds and methods of treating Hepatitis C by the administration of such compounds.

This application claims benefit to provisional application U.S. Ser. No. 60/567,270, filed Apr. 30, 2004; and to provisional application U.S. Ser. No. 60/568,590, filed May 6, 2004; under 35 U.S.C. 119(e). The entire teachings of the referenced applications are incorporated herein by reference.

### FIELD OF THE INVENTION

The present invention includes assays useful for identifying inhibitors of Hepatitis C virus (HCV) activity and for determining the specificity of such inhibitors for HCV. Particularly, the present invention includes a HCV assay useful for high throughput screening that quantifies both the amount of HCV RNA replication inhibitory activity associated with a test compound and the amount of cytotoxicity associated with the test compound, and allows for the measurement of the specificity of the test compound for HCV. As such, an assay of the present invention permits the determination of inhibitory activity associated with a test compound, selectivity of that test compound and specificity of the test compound for HCV in a single well.

### BACKGROUND OF THE INVENTION

Hepatitis C virus (HCV) is the major etiological agent of 90% of all cases of non-A, non-B hepatitis (Dymock, B. W. Emerging Drugs 6:13-42 (2001)). The incidence of HCV infection is becoming an increasingly severe public health concern with 2-15% individuals infected worldwide. While primary infection with HCV is often asymptomatic, most HCV infections progress to a chronic state that can persist for decades. Of those with chronic HCV infections, it is believed that about 20-50% will eventually develop chronic liver disease (e.g. cirrhosis) and 20-30% of these cases will lead to liver failure or liver cancer. As the current HCV-infected population ages, the morbidity and mortality associated with HCV are expected to triple.

Known treatments for HCV infection include the use of interferon-α (IFN), which indirectly effects HCV infection by stimulating the host antiviral response. IFN treatment is largely ineffective, however, as a sustained antiviral response is produced in less than 30% of treated patients. Further, IFN treatment induces an array of side effects of varying severity in upwards of 90% of patients (e.g. acute pancreatitis, depression, retinopathy, thyroiditis). Therapy with a combination of IFN and ribavirin has provided a slightly higher sustained response rate, but has not alleviated the IFN-induced side effects.

One research area of active interest includes the development of antiviral agents which inactivate virally encoded protein products essential for HCV viral replication. Examples of such agents include various tripeptide compounds, which act as selective HCV NS3 serine protease inhibitors. However, many of these compounds do not sufficiently inhibit HCV protease activity or do not have sufficient potency, and thus, may not provide optimal treatment of HCV-infected patients. Accordingly, there is an ongoing need for the development of HCV assays for the identification of agents effective for inactivating viral replication proteins.

Known cell-based assays for screening compounds for HCV inhibitory activity rely upon the detection of viral RNA replication using RT-PCR (Ito et al., Hepatology 34(3):566-572 (2001); Bartenschlager R. and V. Lohman, Antiviral Res. 52(1):1-17 (2001)). Such cell-based systems often yield variable results, making reproducibility a major problem and the use of such system for the screening of compounds impractical, particularly for use in high throughput screening (HTS). HCV assays which rely on the inhibition of viral enzymes essential for viral replication and which may be suitable for HTS are known (Bianchi et al., Analytical Biochemistry 237, 239-244 (1996); Taliani et al., Analytical Biochemistry 240, 60-67 (1996)), but such assays measure only in vitro activity.

Accordingly, there exists a need for an accurate and reproducible cell-based HCV assays which permits the screening of compounds for HCV replication inhibitory activity. The present invention is directed towards such assays.

### SUMMARY OF THE INVENTION

In one aspect, the present invention is directed to a cell-based assay for identifying a compound that inhibits HCV RNA replication and has specificity for HCV. The assay includes the steps of: (a) providing a first cell which expresses at least one enzyme associated with HCV RNA replication; (b) providing a second cell comprising a viral replicon which is not a HCV replicon; (c) contacting the first cell and the second cell with a test compound; (d) determining whether the test compound inhibits HCV RNA replication; (e) determining whether the test compound is cytotoxic to the cell; and (f) determining whether the test compound inhibits the activity of the viral replicon which is not a HCV replicon.

Desirably, the first cell includes a HCV replicon and the second cell comprises a BVDV replicon. The HCV replicon may include a polynucleotide having the nucleic acid sequence set forth in SEQ ID NO:1 and may encode a polypeptide having the amino acid sequence set forth in SEQ ID NO:2. The HCV replicon desirably includes the molecular construct set forth in FIG. 1. The BVDV replicon may include a polynucleotide having the nucleic acid sequence set forth in FIG. 9. Further, the first cell which expresses at least one enzyme associated with HCV RNA replication may be a cell having ATCC Accession No. PTA-4583. The second cell desirably includes a reporter gene, such as luciferase, incorporated within the viral replicon which is not a HCV replicon.

The step (f) of determining whether the test compound is specific for the first cell may be accomplished by measuring the activity of the reporter gene. The enzyme associated with HCV RNA replication may be a protease, particularly a serine protease such as NS3 protease. The step of determining whether the test compound inhibits HCV RNA replication may be conducted by contacting the first cell with a fluorescence substrate, such as a FRET peptide. The step of determining whether the test compound is cytotoxic to the cell may be conducted by contacting the first cell with an Alamar Blue solution. The cell-based assay may be performed in a high-throughput manner.

In another aspect, the present invention is directed to a compound identified by a cell-based assay, above.

In another aspect, the present invention is directed to a pharmaceutical composition including a compound identified by a cell-based assay, above.

In another aspect, the present invention is directed to a method for treating hepatitis-C, including the step of administering to a mammalian species in need thereof a therapeutically effective amount of a compound identified by a cell-based assay, above.

### BRIEF DESCRIPTION OF THE DRAWINGS

FIG. 1 shows the molecular construct of the HCV Replicon used in an assay of the present invention.

FIG. 2 shows the nucleic acid sequence of the HCV Replicon used in an assay of the present invention.

FIG. 3 shows the amino acid sequence of the HCV Replicon used in an assay of the present invention.

FIG. 4 shows the 96-well layout used in an assay of the present invention.

FIG. 5 shows the results of Interferon Titration in the HCV Replicon cell line used in an assay of the present invention.

FIG. 6A shows an EC50 comparison of typical values determined by FRET, RT-PCR or Western analysis for titration of interferon in the HCV replicon cell line.

FIG. 6B shows a Western immunoblot using an anti-NS3 protease serum for the determination of EC₅₀ of IFN-α.

FIG. 7A shows the enzyme activity in each well after contact with test compounds.

FIG. 7B shows the cytotoxicity activity in each well after contact with test compounds.

FIG. 8 shows a graphical representation of the variation within an assay of the present invention.

FIG. 9 shows the nucleic acid sequence of a BVDV Replicon useful in an assay of the present invention.

FIG. 10A shows the layout of compound titration for BVDV and HCV replicon cells in the single 96 well plates shown in FIGS. 10B-10D.

FIG. 10B shows Alamar blue readings for wells treated with BVDV inhibitor, HCV inhibitor or DMSO according to FIG. 10A.

FIG. 10C shows relative HCV FRET rates for wells treated with BVDV inhibitor, HCV inhibitor or DMSO according to FIG. 10A.

FIG. 10D shows relative BVDV luciferase activity for wells treated with BVDV inhibitor, HCV inhibitor or DMSO according to FIG. 10A.

### DETAILED DESCRIPTION OF THE INVENTION

The present invention includes a cell-based HCV assay for measuring the ability of compounds to inhibit HCV RNA replication. An assay of the present invention desirably includes a first cytotoxicity assay step which measures the conversion of an indicator solution to a fluorescent product, to determine if a test compound is cytotoxic to a cell; a second inhibition assay step, to determine if the test compound inhibits HCV RNA replication; and a third specificity step, to determine if the test compound is specific for HCV over other viral replicons. Desirably, an assay of the present invention include the use of cells transfected with a HCV replicon and cells transfected with a BVDV replicon. The BVDV replicon incorporates a reporter gene, such as luciferase.

The ability of the HCV replicon to replicate is highly dependent on the amounts or activity of host cell factors. Therefore, any slight toxicity may have significant effects on viral protein expression and ultimately on any assay which examines the effect of compounds on HCV replication. As such, the use of an indicator to assess cytotoxicity in an HCV replicon cell line in an assay of the present invention provides a significant advantage in the ability to address the issue of whether HCV inhibition is due to a specific compound-virus interaction or due to a subtle but toxic effect on the cellular replication machinery. Moreover, the present invention includes determining the specificity of a test compound for HCV, desirably in the same well as inhibitory activity and cytotoxicity are determined. Specificity may be determined by introducing the test compound to a cell comprising a Bovine viral diarrhea virus (BVDV) replicon (a closely related virus to HCV) which incorporates a reporter gene. Test compounds specific for HCV inhibition do not substantially inhibit BVDV activity, as measured by luciferase activity.

Accordingly, the present invention includes an assay useful for HTS that quantifies the amount of HCV RNA replication inhibitory activity associated with a test compound and the amount of cytotoxicity associated with that test compound, and further measures the specificity of the test compound for HCV. The inventive assay is desirably conducted in a single well. Assays of the present invention permit for the mass screening of compounds specifically directed towards HCV replication, and permit viral RNA as well as viral proteins to be produced at levels consistently detectable using standard immunological and molecular biology methods. These consistent levels are amendable for HTS of compounds specific for the HCV replicon since effects either toxic to the cell or specific to the replicon can be differentiated and quantitated.

In an assay of the present invention, a first cytotoxicity assay step measures the conversion of an Alamar Blue solution to a fluorescent product, a second inhibition assay step that uses a fluorescence resonance energy transfer (FRET) protease substrate specifically measures the amount of HCV NS3 protease activity present and relates that activity to HCV RNA amounts, and a third specificity step measures the expression of a reporter construct which is incorporated into a BVDV replicon to determine the specificity of a test compound for HCV. The first cytotoxicity assay step permits the determination of selectivity of the test compound under consideration for the cells in the assay. The use of Alamar Blue solution permits the assay steps to be run in the same well, as the Alamar Blue solution is non-lethal to the cells.

An assay of the present invention has been validated and compared with quantitative reverse-transcriptase polymerase chain reaction (qRT-PCR) and western blot analysis using interferon-α, a known HCV inhibitor. An assay of the present invention yielded fifty-percent effective concentration (EC50) values of 1.9, 2.9 and 5.3 units for the western, FRET and qRT-PCR assays, respectively. Assay of the present invention are amenable for HTS to identify compounds which inhibit HCV RNA replication, providing a convenient and economical assay comparable to qRT-PCR.

HCV is a plus (+) strand RNA virus which is well characterized, having a length of approximately 9.6 kb and a single, long open reading frame (ORF) encoding an approximately 3000-amino acid polyprotein (Lohman et al., Science 285:110-113 (1999), expressly incorporated by reference in its entirety). The ORF is flanked at the 5′ end by a nontranslated region that functions as an internal ribosome entry site (IRES) and at the 3′ end by a highly conserved sequence essential for genome replication (Lohman, supra). The structural proteins are in the NH₂-terminal region of the polyprotein and the nonstructural proteins (NS) 2 to 5B in the remainder.

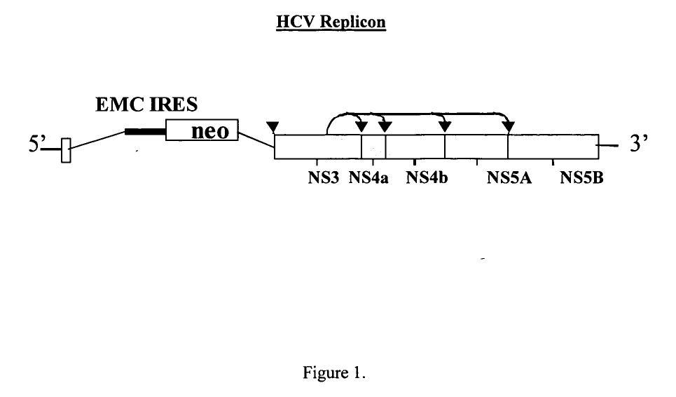

In an assay of the present invention, a HCV replicon was used in a cell culture system and was made as set forth below in Materials and Methods. A bovine viral diarrhea virus (BVDV) replicon was also made as set forth below in Materials and Methods. The HCV replicon was based on a full-length consensus genome cloned from viral RNA isolated from an infected human liver. As shown in the molecular construct set forth in FIG. 1, a HCV replicon useful in an assay of the present invention includes a neomycin (neo) selectable marker protein translated from the native HCV internal ribosome entry site (IRES) element and non-structural proteins translated by the IRES from encephalomyocarditis virus (Lohman, supra). The known viral specific enzymatic activities provided by the replicon include the protease (NS3) and activator of the protease (NS4A), helicase (NS3), ATPase (NS3) and RNA dependent RNA polymersase (NS5B). Expression of neo is solely dependent on active HCV RNA replication in cells, and the viral gene products NS3 to NS5B are believed to be essential for HCV RNA replication and are the primary targets for inhibitor identification. For purposes of the present invention, viral gene products which are “associated” with HCV RNA replication include any and all viral gene products believed to be essential for HCV RNA replication.

Methods used to quantitate HCV can be applied to the replicon and include quantitative RT-PCR (qRT-PCR) for RNA levels and immunological methods for proteins such as ELISA (Rodriguez-Lopez et. al., J. Gen. Virol. 80:727-738 (1999), expressly incorporated by reference in its entirety) or Western analysis (Pietschmann et al., J. Virol. 75:1253-1264 (2001), expressly incorporated by reference in its entirety).

An assay of the present invention desirably consists of at least three parts. The first part is a cytotoxicity assay step which quantitates the amount of cytotoxicity associated with a test compound, as determined by the conversion of Alamar Blue dye. The second part is an inhibition assay step which quantitates the amount of NS3 protease activity associated with the test compound. The third part is a specificity step in which the ability of a test compound to inhibit BVDV activity is determined by reporter expression. A test compound which is specific for HCV inhibition will not inhibit BVDV activity. All measurements are compared relative to control wells. The inventive methods provide a measure of cytotoxicity for each well, an indirect measure of HCV RNA levels and a determination of the specificity of the test compound for HCV inhibition.

Inhibition of HCV RNA replication is expected to reduce the amount of viral proteins present, including NS3 protease. As such, inhibitory activity of test compounds on HCV RNA replication is indirectly measured by quantitating NS3 protease levels using a FRET assay. The results obtained with the FRET assay have been shown to be comparable to those obtained from qRT-PCR.

The following section sets forth materials and methods used in the present invention, and which were utilized in the Examples set forth hereinbelow.

### Materials and Methods

1. HCV Replicon Cell Line Preparation

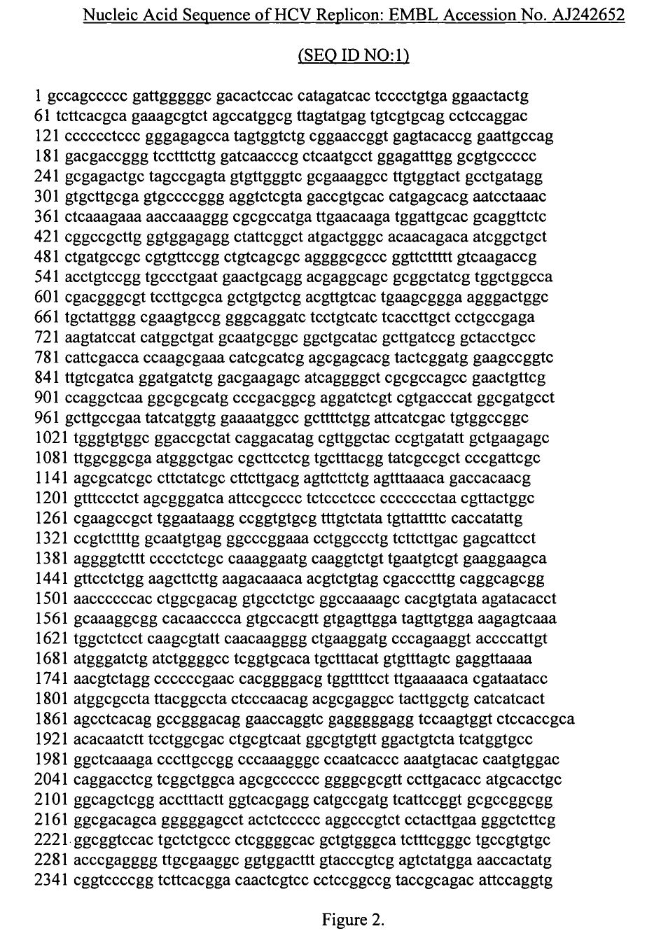

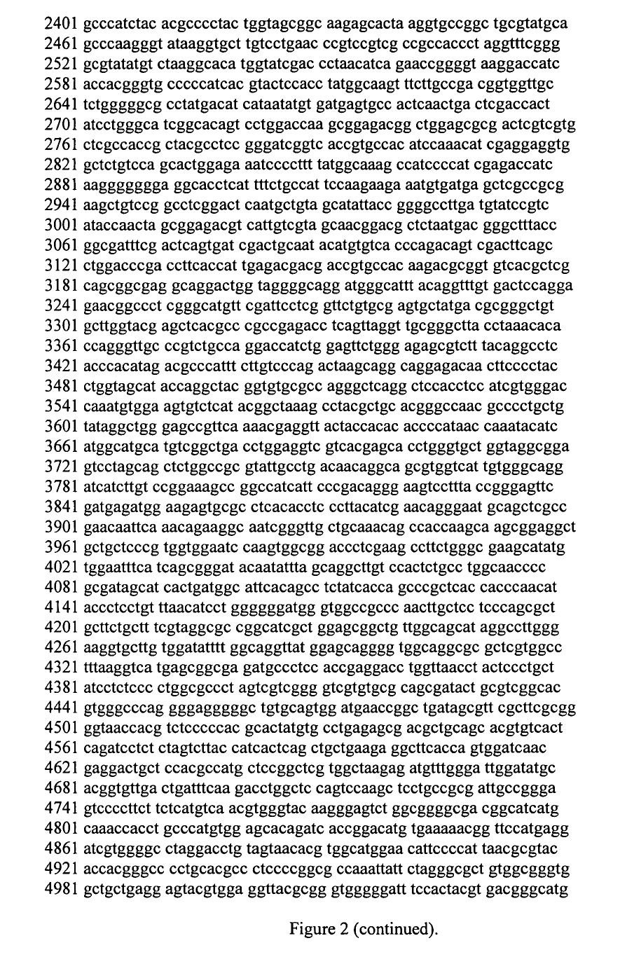

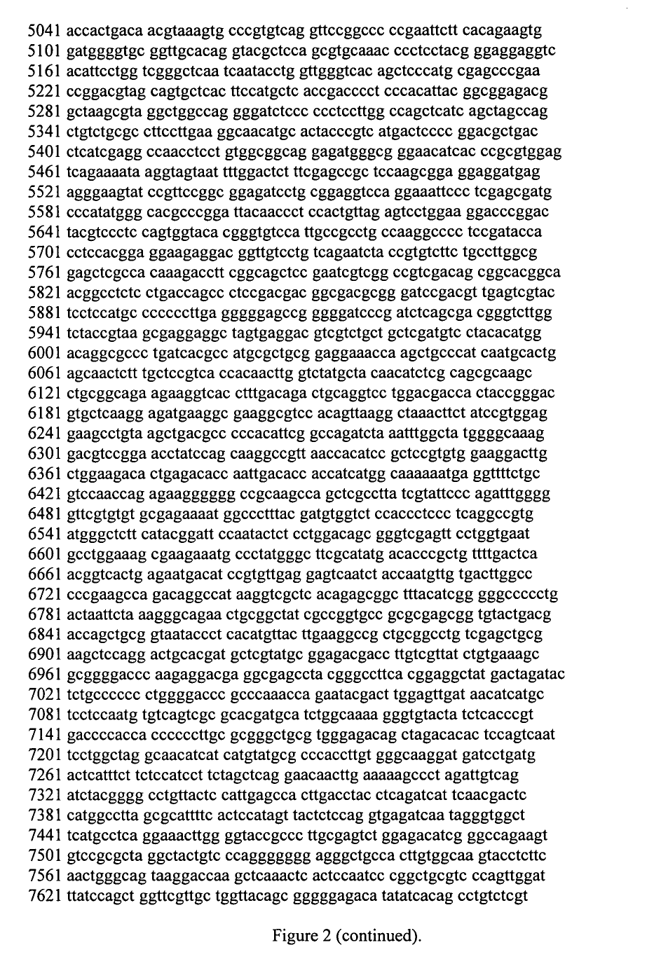

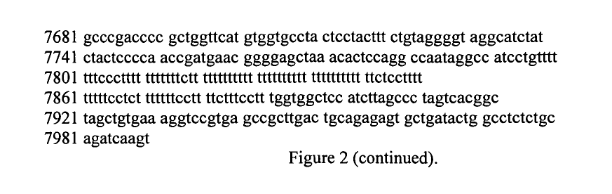

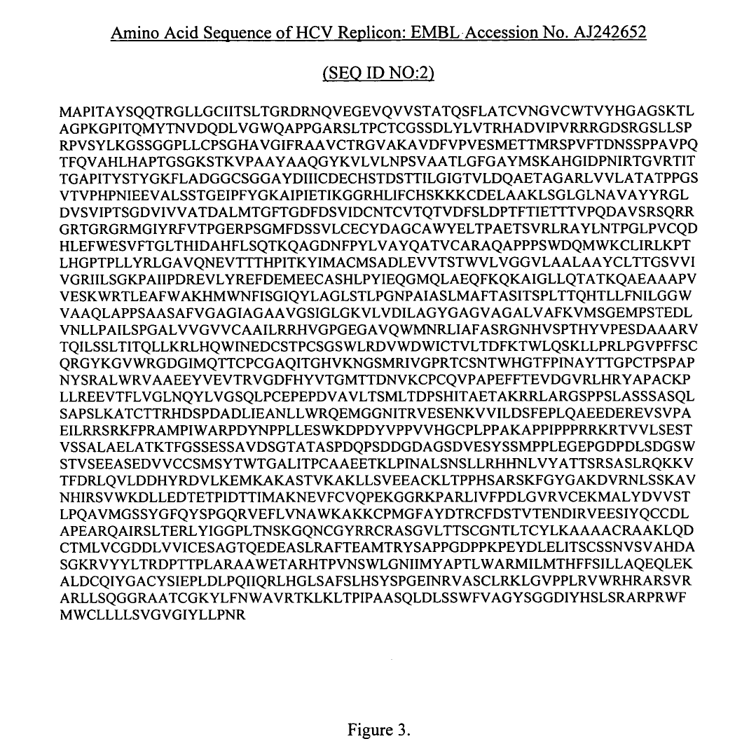

The HCV replicon cell line was isolated from colonies as described by Lohman et. al. (Lohman, supra) and used for all experiments. The HCV replicon has the nucleic acid sequence set forth in FIG. 2 (EMBL Accession No.: AJ242652; SEQ ID NO:1), the coding sequence of which is from 1801nt-7758nt. The coding sequence encodes the polypeptide having the sequence set forth in FIG. 3 (SEQ ID NO:2).

The cell line used in the present invention has been deposited as ATCC Accession No. PTA-4583 in the American Type Culture Collection, 10801 University Boulevard, Manassas, Va. 20110-2209 U.S.A. under the terms of the Budapest Treaty on the International Recognition of Deposits of Microorganisms for Purposes of Patent Procedure and the Regulations promulgated under this Treaty. Samples of the deposited material are and will be available to industrial property offices and other persons legally entitled to receive them under the terms of the Treaty and Regulations and otherwise in compliance with the patent laws and regulations of the United States of America and all other nations or international organizations in which this application, or an application claiming priority of this application, is filed or in which any patent granted on any such application is granted.

The coding sequence of the published HCV replicon was synthesized by Operon Technologies, Inc. (Alameda, Calif.), and the full-length replicon was then assembled in plasmid pGem9zf(+) (Promega) using standard molecular biology techniques. The replicon consists of (i) the HCV 5′ UTR fused to the first 12 amino acids of the capsid protein, (ii) the neomycin phosphotransferase gene (neo), (iii) the IRES from encephalomyocarditis virus (EMCV), and (iv) HCV NS3 to NS5B genes and the HCV3′ UTR. Plasmid DNAs were linearized with ScaI and RNA transcripts were synthesized in vitro using the T7 MegaScript transcription kit (Ambion) according to manufacturer's directions.

To generate cell lines, 4×10⁶ Huh-7 cells (kindly provided by R. Bartenschlager and available from Health Science Research Resources Bank, Japan Health Sciences Foundation) were electroporated (GenePulser System, Bio-Rad) with 10 ug of RNA transcript and plated into 100-mm dishes. After 24 h, selective media containing 1.0 mg/ml G418 was added and media was changed every 3 to 5 days. Approximately 4 weeks after electroporation, small colonies were visible which were isolated and expanded for further analysis. These cell lines were maintained at 37° C., 5% CO₂, 100% relative humidity in DMEM (Life Technologies #11965-084) with 10% heat inactivated calf serum (Sigma #F-2442), 100 U/ml of penicillin/streptomycin (Life Technologies #15140-122), Geneticin at 1 mg/ml (Life Technologies #10131-027). One of the cell lines which had approximately 3,000 copies of HCV replicon RNA/cell was used for development of the assay.

Other HCV replicons, as well as different genotypes, are suitable for use in assays of the present invention, and it is to be understood that assay of the present invention are not limited to any particular HCV replicon or cell line created therefrom. For example, in addition to the HCV replicon described above, HCV replicons suitable for use in assays of the present invention include, but are not limited to, those available from Apath, LLC. Also, it is understood that modifications of such HCV replicons may be made such that the replicon is useful in assays of the present invention.

2. BVDV Replicon Cell Line Preparation

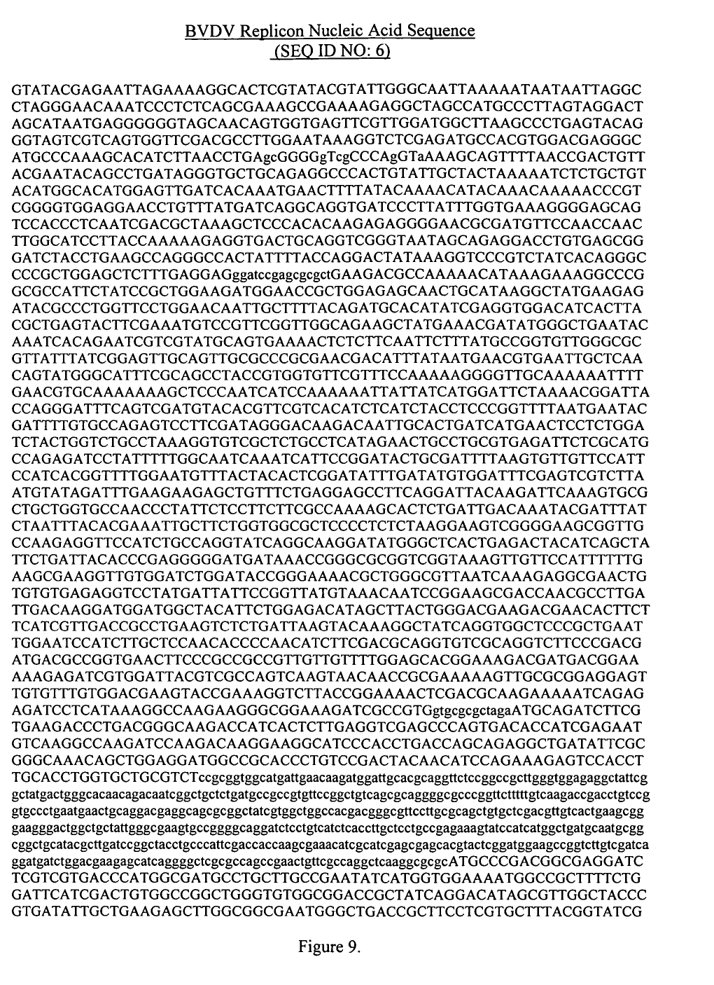

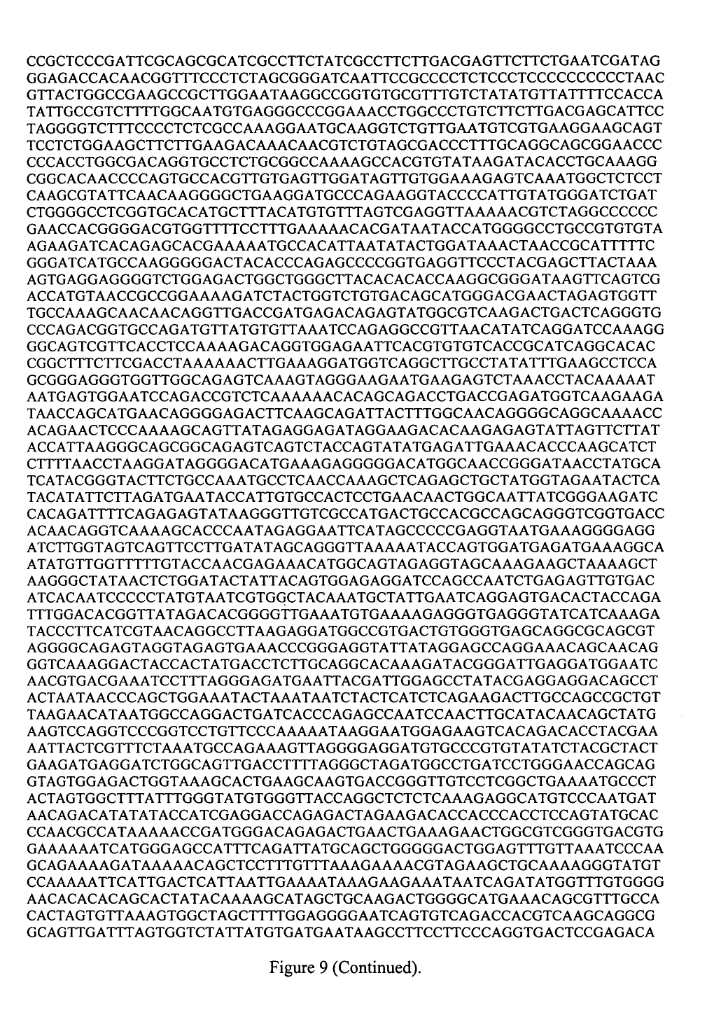

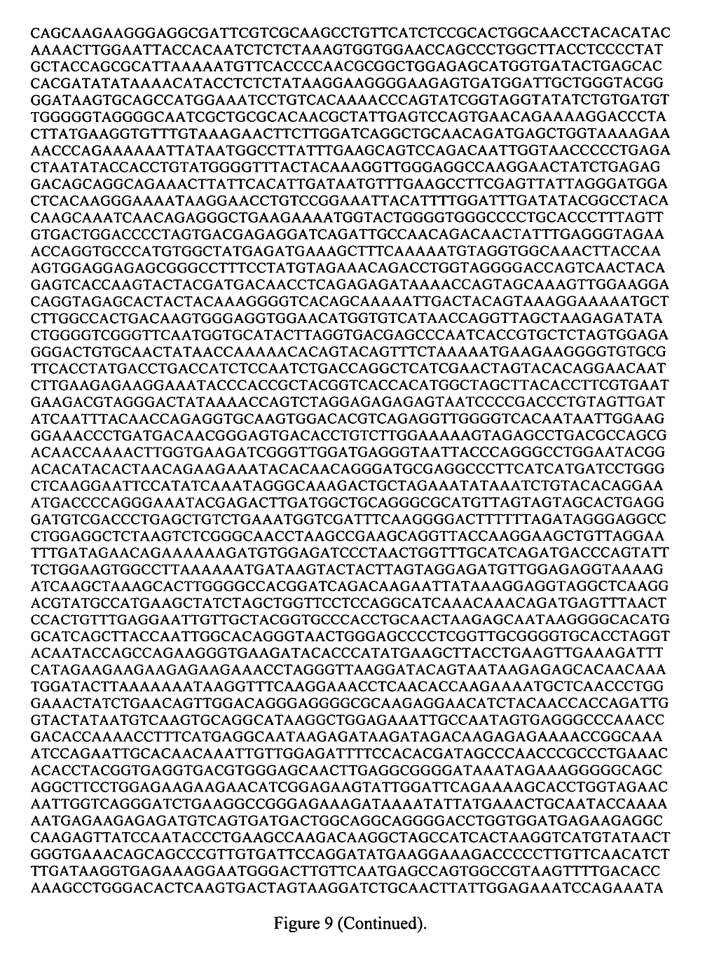

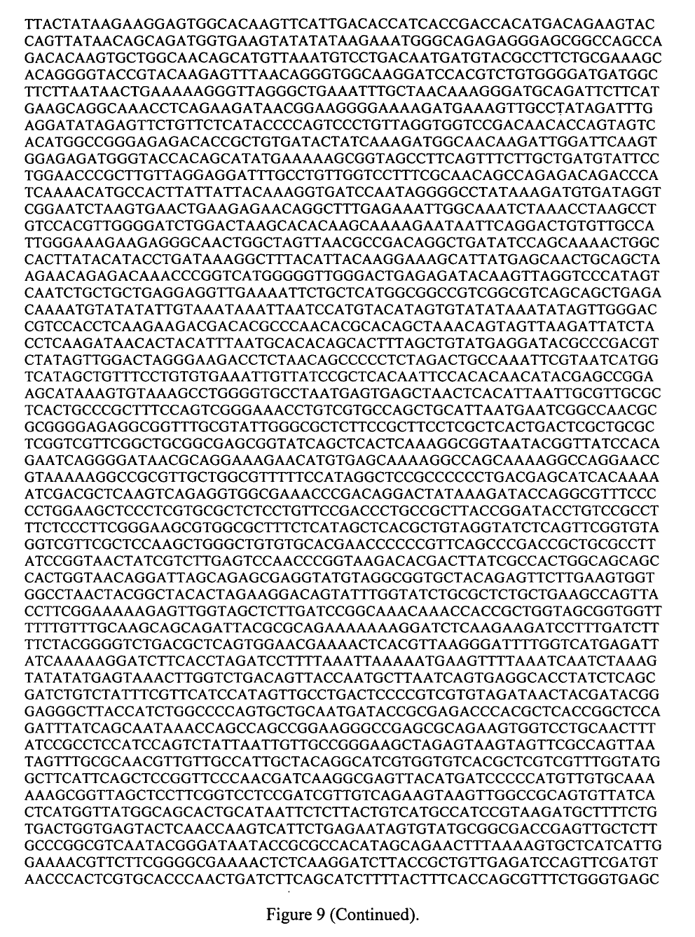

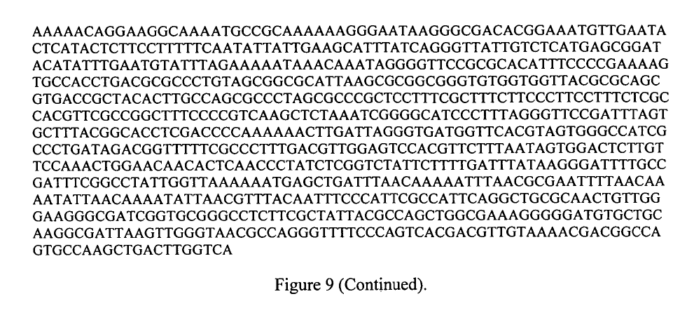

To generate a BVDV replicon (termed BVDV-bu; Nucleic acid sequence shown in FIG. 9; SEQ ID NO: 6), the SphI/BglII fragment from 153E-2 (Sun, Jin-Hua et al., J. Virol. (2003) 77(12):6753-60, specifically incorporated herein by reference) was ligated with the SphI/BssHII fragment from 166A-4 (Sun et al., supra), a BssHII/SacII fragment from ubiquitin and a SacII/BglII digested PCR fragment which was the BVDV NS3 region amplified to incorporate the C-terminus of ubiquitin onto the 5′ end of NS3. A firefly luciferase gene was then amplified by standard PCR methods to add BssHII sites at each end and cloned into BVDV-bu at a BssHII site at nt 740 by nondirectional cloning to generate BVDV-Luc. The neomycin gene and EMC IRES were PCR amplified from the HCV genotype 1b replicon plasmid and ligated into BVDV-Luc to generate the final clone (BVDV-Luc-neo) consisting of the BVDV 5′ UTR followed by the gene for firefly luciferase, a ubiquitin monomer, the neomycin phosphotransferase gene, the EMCV IRES, BVDV NS3-5B and the BVDV 3′ UTR.

Stable BVDV-Luc-neo cell lines were generated and maintained as described above using 0.5 mg/ml G418 selection. BVDV RNA levels in these cell lines were examined directly using quantitative Taqman RT/PCR and BVDV proteins were confirmed by Western blot. In addition, the BVDV luciferase assay was validated in these cell lines by examining luciferase levels in the presence and absence of compound-1453, a specific inhibitor of BVDV replication (Sun et al., supra). As determined by luciferase, the EC₅₀ of compound-1453 was ˜1 uM, which is comparable to previous results obtained with BVDV virus (Sun et al., supra).

3. RNA Detection

HCV RNA detection was conducted using RT-PCR, according to the manufacturer's instructions, using a Gibco-BRL Platinum Quantitative RT-PCR Thermoscript One-Step Kit on a Perkin-Elmer ABI Prism Model 7700 sequence detector. The primers for TaqMan were selected for use following analysis of RNA sequences with Primer Express Software from ABI. Primers used for detection of the plus strand RNA were 131F -5′ GGGAGAGCCATAGTGGTCTGC 3′ (SEQ ID NO:3) and 231R- 5′ CCCAAATCTCCAGGCATTGA 3′ (SEQ ID NO:4) which amplify the HCV 5′UTR from nucleotides 131 to 231. The probe used for detection, 5′FAM-CGGAATTGCCAGGACGACCGG-BHQ1 3′ (SEQ ID NO:5) was obtained from Biosearch Technologies. RNA's were purified from 96-wells using the RNAeasy 96 kit from Qiagen.

4. Western Analysis

Experiments were done in duplicate. Western analysis was performed according to the instructions for Amersham's Chemiluminescence Immunology Kit (NEL105 Renaissance) using a Molecular Dynamics Storm 860 phosphoimager and associated software. The primary and secondary antibody dilutions were at 1 to 5,000. Antisera was generated by immunizing rabbits with purified NS3 protease made from an E. Coli expression vector encoding the first 181 amino acids of HCV 1a NS3 with subsequent boosts.

Bleeds were tested weekly and boosts continued until a positive signal on a control western was seen. Secondary antibody was a BioRad (#170-6515) Goat anti-Rabbit IgG HRP Conjugate. The protein samples for western analysis were from the same wells used for the FRET assay and were prepared by the addition of an equal volume of 2× SDS-PAGE buffer to the FRET assay mixture, heating and loading on a 10% gel for SDS-PAGE. Interferon alpha (IFN-α) was obtained from Sigma (#I-4276) and stored as recommended.

5. FRET Assay Preparation

To perform the HCV FRET screening assay, 96-well cell culture plates were used. The FRET peptide (Anaspec, Inc.) (Taliani et al., Anal. Biochem. 240:60-67 (1996), expressly incorporated by reference in its entirety) contains a fluorescence donor, EDANS, near one end of the peptide and an acceptor, DABCYL, near the other end. The fluorescence of the peptide is quenched by intramolecular resonance energy transfer (RET) between the donor and the acceptor, but as the NS3 protease cleaves the peptide the products are released from RET quenching and the fluorescence of the donor becomes apparent.

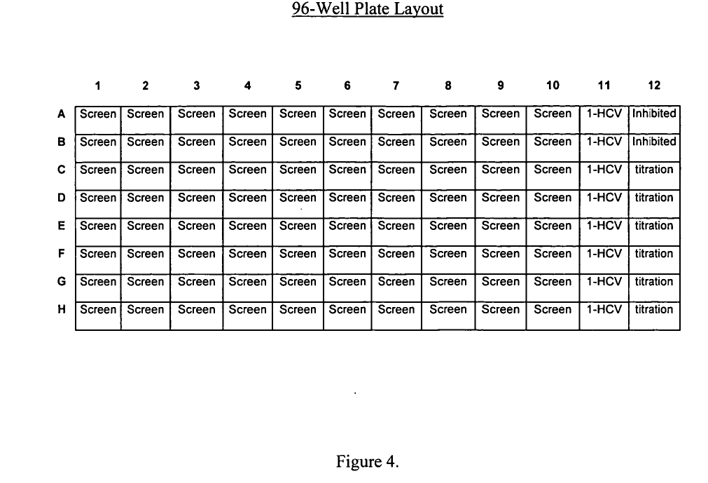

The assay reagent was made as follows: 5× cell Luciferase cell culture lysis reagent from Promega (#E153A) diluted to 1× with dH₂O, NaCl added to 150 mM final, the FRET peptide diluted to 20 uM final from a 2 mM stock. Cells were trypsinized, placed into each well of a 96-well plate and allowed to attach overnight. The next day, the test compounds were added to columns 1 through 10; column 11 was media only, and column 12 contained a titration of interferon as a control (1000 units for A12, B12, 100 units for C12, D12, 10 units for E12, F12 and 1 unit for G12, H12). In addition, replicon cells in A12, B12 can be replaced, if desired, with naive Huh-7 cells as a negative background control. The plates were then placed back in the incubator. FIG. 4 shows the layout for HTS of the replicon cell line in a 96-well plate. In FIG. 4, labels are as followed: “Screen” denotes wells with test compound; “1-HCV” denotes control replicon wells (100% activity), “Inhibited” denotes wells containing the highest amount of control inhibitor (100% inhibition), and was used to determine background for each plate; “Titration” denotes the titration of interferon, and was used as a sensitivity control. Units of interferon from the top of row 12 in duplicate are 1000, 100, 10, and 1.

6. FRET Assay and Cytotoxicity Assay Steps

Subsequent to addition of the test compounds described above (FRET Assay Preparation), at various times the plate was removed and Alamar Blue solution (Trek Diagnostics, #00-100) was added per well as a measure of cellular toxicity. After reading in a Cytoflour 4000 instrument (PE Biosystems), plates were rinsed with PBS and then used for FRET assay by the addition of 30 ul of the FRET peptide assay reagent described above (FRET Assay Preparation) per well. The plate was then placed into the Cytoflour 4000 instrument which had been set to 340 excite/490 emission, automatic mode for 20 cycles and the plate read in a kinetic mode. Typically, the signal to noise using an endpoint analysis after the reads was at least three-fold.

Compound analysis was determined by quantification of the relative HCV replicon inhibition and the relative cytotoxicity values. To calculate cytotoxicity values, the average Alamar Blue fluorescence signals from the control wells in row 11 (FIG. 4) were set as 100% non-toxic. The individual signals in each of the compound test wells were then divided by the average control signal and multiplied by 100% to determine percent cytotoxicity. To calculate the HCV replicon inhibition values, an average background value FRET signal was obtained from the two wells containing the highest amount of interferon at the end of the assay period. These numbers were similar to those obtained from naïve Huh-7 cells.

The background numbers were then subtracted from the average FRET signal obtained from the control wells in row 11 (FIG. 4) and this number was used as 100% activity. The individual signals in each of the compound test wells were then divided by the averaged control values after background subtraction and multiplied by 100% to determine percent activity. EC₅₀ values for an interferon titration were calculated as the concentration which caused a 50% reduction in HCV RNA, HCV protein amounts or FRET activity. The two numbers generated for the compound plate, percent cytotoxicity and percent activity were used to determine compounds of interest for further analysis.

7. Calculation of Assay Variation

The following formula was used to calculate the variation in the FRET assay. Z′ is a measure of the distance between the standard deviations for the signal versus the noise of the assay:
Z′=1−((3*asds+3*asdb)/(as−ab))
Asds=standard deviation of the signal
Asdb=standard deviation of the background
As=average signal
Ab=average background signal
(Zhang et al., J. Biomolecular Screening (4) 2:67-73 (1999), expressly incorporated by reference in its entirety).

### EXAMPLE 1

### HCV Inhibition and Cytotoxicity Measurements

An assay of the present invention was prepared and conducted in the manner set forth above in Materials and Methods. The HTS assay was designed to indirectly measure RNA levels through the use of a specific NS3 protease fluorescence substrate which yields a fluorescent signal upon cleavage. To ensure that the NS3 protease substrate could only be cleaved by the NS3 protease and not by any cellular proteases present in the replicon cell lysates, the substrate was added to individual wells containing crude lysates made from either naive Huh-7 cells, HepG-2 cells or HeLa cells. The substrate was found to only yield a substantial increase in fluorescence in cells containing either the HCV replicon or in cells expressing the NS3 enzyme, indicating that the assay was specific for HCV protease.

Prior to the FRET assay step, a solution of Alamar Blue was added to the same plates in a cytotoxicity assay step, allowing direct quantification of the level of toxicity in that well. Only compounds which show no apparent toxicity but significantly decrease the amount of NS3 protease activity were further analyzed for HCV inhibitory activity.

In order to validate the FRET assay for HTS, the relationship between viral RNA levels and the amount of NS3 activity present was quantitated. One consideration of using the NS3 protease as a general indicator of RNA levels is that the t_{1/2} life of the RNA compared to the protein may be substantially different (Lohman, supra). This could result in a substantial drop in RNA levels rather quickly compared to protein amounts. To compensate for this difference, the cells were exposed to interferon alpha (IFN-α), a known HCV inhibitor (Lauer G. M. and B. D. Walker, N. Engl. J. Med. 345(1):41-52 (2001); Blight et al., Science, 290:1972-1974 (2000); Collier J. and R. Chapman, BioDrugs, 15(4):225-238 (2001), each of which is expressly incorporated by reference in its entirety), for a period of days, allowing the cells to magnify the effect and let the amount of NS3 present decrease relative to controls.

The validation of the assay was accomplished by the use of quantitative RT-PCR (qRT-PCR) for viral RNA levels, quantification of the amount of NS3 present by scanning of a Western blot for protein levels and measurement of NS3 protease activity using the FRET assay. The samples for these measurements were from 2 plates prepared the same day and treated at the same time with a titration of IFN-α. One plate was used for preparation of RNA for quantitative RT-PCR while the other plate was used for FRET. Samples from the same wells after the FRET assay were used for Western analysis. Compound plates were then used to ensure that the procedure was applicable under conditions of HTS.

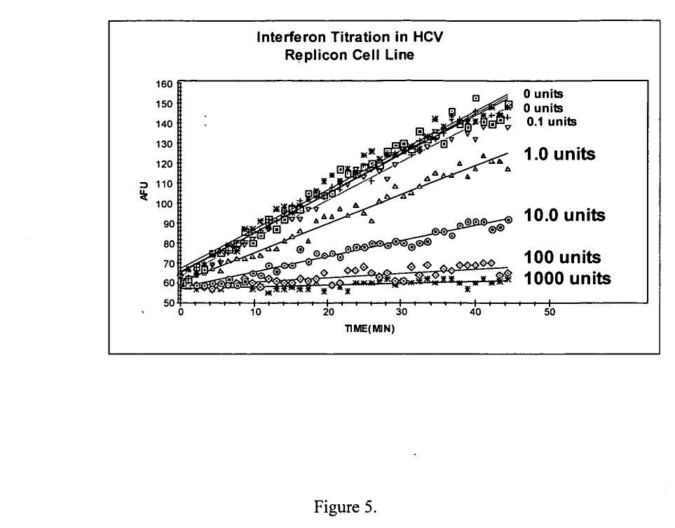

The results of a FRET assay with IFN-α titration following 96 hours of incubation are shown in FIG. 5 as a continuous kinetic graph. FIG. 5 shows the measurement of the increase in fluorescence of the HCV FRET peptide in the HCV cell line and the effect of exposure to various interferon concentrations. The units per ml of IFN-α used for the different wells are listed to the right of the pertinent graphs. The assay is linear over a period of 40 minutes. As seen in FIG. 5, in the absence of IFN-α, the FRET signal is increased with time and is linear for at least 30 minutes. A decrease in the rate of FRET activity is clearly evident in the graph with increasing IFN-α concentration. The titration was from 0.1 units to 1,000 units per milliliter with control wells containing IFN-α dilution buffer only.

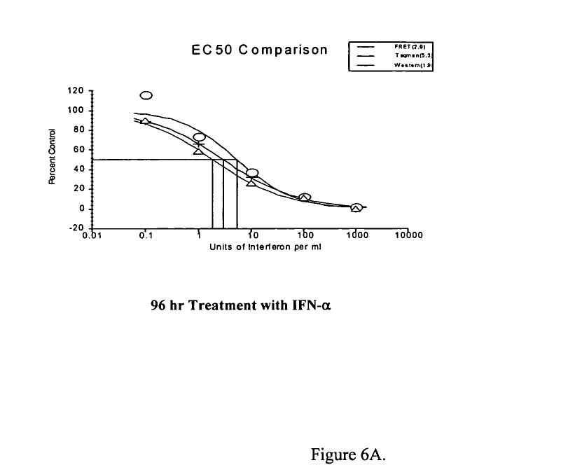

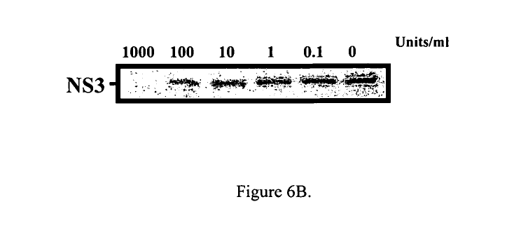

Calculations involved subtracting the final background fluorescence signal while using the control wells as 100% activity. These numbers from the linear range are required for determination of the IFN-α EC₅₀. Similarly, RNA levels were measured by qRT-PCR while the amount of NS3 protein present in each well was quantitated by scanning a Western immunoblot. An EC₅₀ was determined for all three methods by normalizing to the controls for each measurement. FIG. 6A shows a comparison of typical values determined by FRET, RT-PCR or scanning of a western blot for titration of interferon in the HCV replicon cell line, and also shows values for quantification of NS3 protease specific bands (FIG. 6B) by phosphorimaging. Each value in FIG. 6A represents a well of a 96-well plate at a single interferon concentration relative to a control value. Data at the lowest concentration of interferon tended to contain more variation. FIG. 6B shows the Western immunoblot using an anti-NS3 protease serum for the determination of EC₅₀ of IFN-α.

The results shown in FIG. 6A indicate EC₅₀ values (in units of IFN-α per milliliter) of 1.9 for the Western, 2.9 for the FRET and 5.3 for RT-PCR. These values are within 3-fold of one another and indicate equivalency between the assay methods. This demonstrates the utility of the FRET assay method for inhibitor titration in an assay of the present invention and provides a comparison of a HTS format to the conventional qRT-PCR method of HCV quantification.

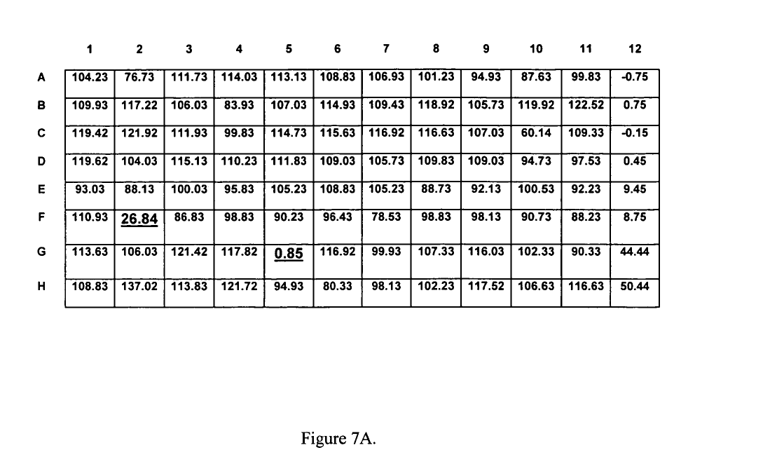

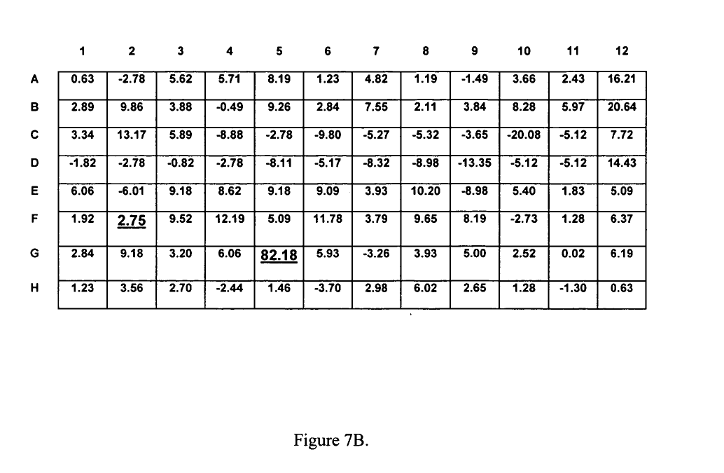

A random compound plate was used in a method test of both the Alamar Blue assay and the FRET HCV replicon assay steps. The results are presented in FIGS. 7A and 7B for both the FRET and Alamar Blue assay as diagrammed in FIG. 4. FIG. 7A shows the percentage of activity in each well following FRET readings and performing the calculations described above for the endpoint reading from cycle 21 of the FRET assay. In FIG. 7A, lower numbers represent less activity present and indicate that the HCV replicon is inhibited. Wells F2 and G5 (underlined and enlarged) indicate that the compounds present in these wells inhibited the HCV replicon approximately 73% and 99% respectively.

FIG. 7B shows Alamar Blue readings from the random compound plate expressed as a measure of cytotoxicity. Wells corresponding to F2 and G5 (underlined and enlarged) indicate that compound present in F2 shows very little toxicity while compound in G5 has substantial toxicity. Comparing the results of the FRET assay with the Alamar assay it is likely that the inhibition of the HCV replicon for G5 is due to a toxic mechanism while the inhibition due to compound in F2 is not toxic in this assay, suggesting the compound may be specific for HCV.

In general, the majority of compounds did not cause a significant variation in either the FRET or Alamar Blue assay indicating acceptable results amenable to HTS. The FRET activity yielded a 12.7% standard deviation in wells containing control media (FIG. 7A, column 11). In the IFN-α control samples, a clear inhibition was observed, the EC₅₀ was close to or slightly lower than the lowest concentration of IFN-α used (FIG. 7A, column 11). The Alamar Blue measurements in this plate yielded a variation of 4% for the cytotoxicity measurements in wells containing control media (FIG. 7B, column 11). Approximately 18% cytotoxicity was observed in the wells with the highest concentration of IFN-α (1000 units, FIG. 7B, columns A12 and B12), but no apparent Alamar Blue staining change was seen at lower concentrations of IFN-α. In the compound test area, two compounds showed a noticeable reduction in FRET activity, down to 27% and 1% detectable activity, respectively, of the control level (FIG. 7A, columns F2 and G5).

Inspection of the numbers and comparison of FIGS. 7A and 7B indicate a toxic compound is present in well G5 due to the decrease in FRET activity along with a corresponding decrease for the Alamar assay. Well F2, however, was seen to have a noticeable decrease in FRET activity without a corresponding decrease in the Alamar Blue measurement, indicating HCV replicon inhibition without measurable toxicity for this compound. Therefore, this compound was chosen for further evaluation.

To confirm that the variation in the FRET assay would remain acceptable, 40 additional compound plates were used to quantitate the variation using a statistical analysis to measure the Z′ statistic (Materials and Methods). The Z′ statistic is a measure of the distance between the standard deviations for the signal versus the noise of the assay. This analysis was used since the signal to noise in the assay was usually only 3-fold which is less than the Alamar signal to noise of approximately 8-fold indicating less tolerance for variation in the assay. An assay is considered acceptable if the Z′ statistic is 0.5 or greater indicating acceptable signal to noise scatter in the plates.

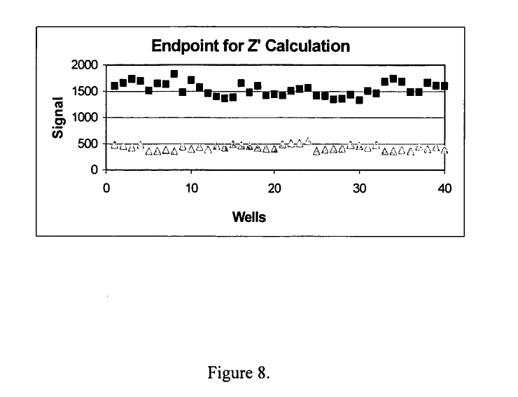

Forty plates were used to measure the standard deviations and the number distribution between the endpoint signal obtained for the controls and the signal obtained for the background. FIG. 8 shows a graphical representation of the averaged numbers from 40 separate compound plates used in the Z′ calculation. The numbers at a signal of approximately 500 are the readings from the wells containing 1000 units of interferon and are considered to have 0% FRET activity. The numbers at a signal of approximately 1500 are from wells containing buffer only and are considered as 100% FRET activity. The Z′ measurement calculates the distance as a fraction between the two number distributions in terms of the means of those distributions.

Using this calculation, a Z′ of 0.62 was obtained indicating a plate to plate variation acceptable for HTS. In addition, this measurement can be used on individual plates to determine if the controls were acceptable validating the data for a particular plate.

### EXAMPLE 2

### HCV/BVDV Dual Assay

BVDV (Bovine viral diarrhea virus) replicon, a closed related virus to HCV, was used for compound specificity evaluation. The BVDV replicon cell line was prepared as stated above. Specificity evaluation was conducted by using a mixed-cell assay format consisting of HCV and BVDV replicon containing Huh-7 cells placed in the same well on a test plate. The complete assay consisted of measuring Alamar blue conversion and quantifying HCV FRET peptide activity (as described above) and then measuring luciferase activity by use of a luciferase reporter gene incorporated into the BVDV genome.

BVDV inhibition was indirectly measured following the Alamar Blue assay and the HCV FRET assay described above by measuring the amount of luciferase activity present in each well. A luciferase substrate (Promega Kit for firefly luciferase #E4550) was added to the wells containing the FRET peptide/lysis solution and the plate placed into a Top Count (Packard Instruments) programmed for luciferase measurements. The percent of BVDV inhibition was quantified relative to a specific BVDV test compound placed into columns of the plate; compound 1453 (Sun et. Al supra.): while columns containing DMSO only were used as 100% luciferase activity. The concentration of compound 1453 was chosen so that the highest dilution used inhibited BVDV 100%, was non-toxic to the cells and did not affect HCV replication (10 uM).

The luciferase amount from wells 100% inhibited were averaged and used as the background luciferase value and were subtracted from all wells before calculating percent luciferase activity. These measurements enabled the prioritization of compounds for further study and resulted in three assay steps being performed in each well of the tissue culture plate: (1) the toxicity was determined by Alamar blue conversion; (2) the HCV inhibition amount by NS3 FRET peptide cleavage; and (3) the amount of BVDV inhibition by luciferase activity. These three measurements determined which compounds were non-toxic, inhibited HCV and were specific for HCV, respectively.

### EXAMPLE 3

### Demonstration of Assay Format Using Known Specific Inhibitors of HCV and BVDV

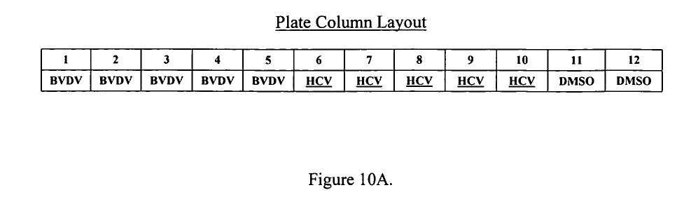

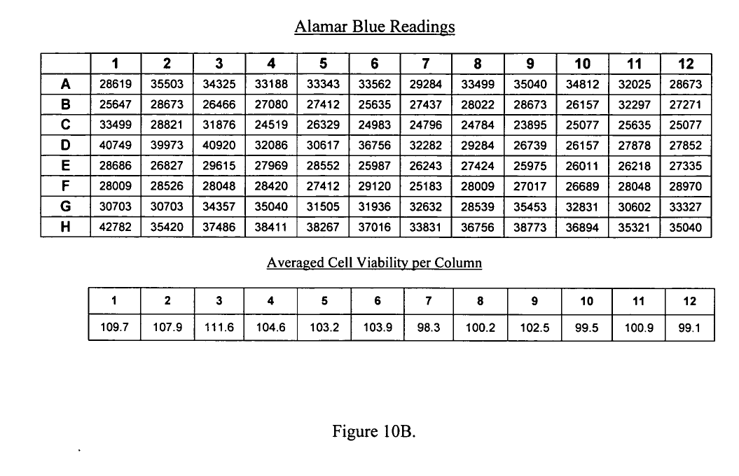

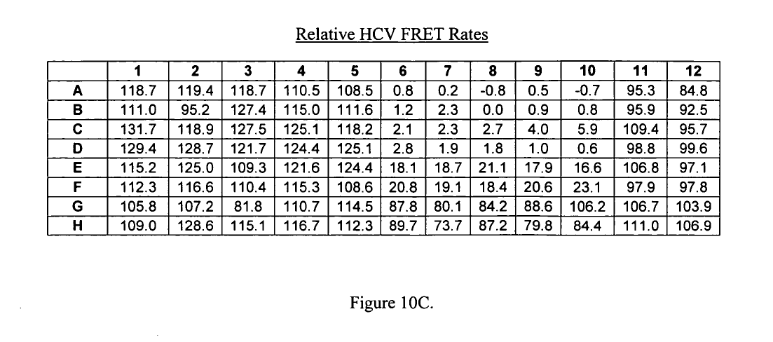

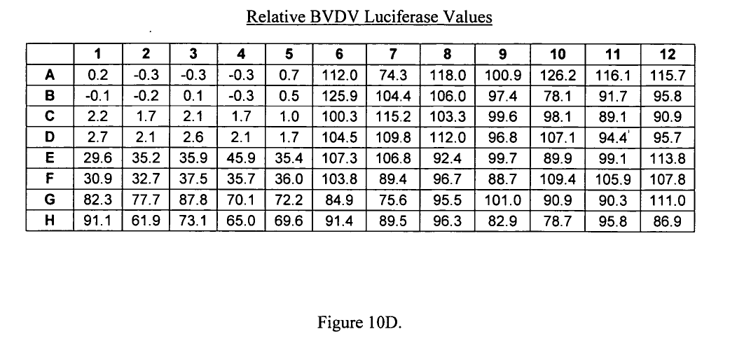

An assay of the present invention was performed as follows: Alamar Blue was added to media and plate returned to incubator; plate was removed after 5 hours; plate was read for Alamar conversion and then washed; HCV FRET assay was added and plate read; BVDV luciferase substrate was added and activity was measured. In FIGS. 10B-10D respectively: (1) toxicity measurements are shown using Alamar blue; (2) HCV inhibition is shown using FRET relative rate numbers; and (3) BVDV inhibition is shown using luciferase reporter gene relative numbers.

FIG. 10A shows the layout of compound titration for BVDV and HCV replicon cells in the single 96 well plates shown in FIGS. 10B-10D. Compounds were titrated from top to bottom in 5 columns each with the highest concentrations in row A. “BVDV” represents titration of compound-1453 in columns 1-5 and “HCV” represents titration of the HCV specific compound (a protease inhibitor, Campbell, J. A., Good, A. C. (Bristol-Myers Squibb Co.) set forth in WO 02/60926, specifically incorporated herein by reference) in columns 6-10. “DMSO” represents Dimethyl Sulfoxide which was added to the wells in columns 11-12 as a control and represents 100% control.

FIG. 10B shows Alamar blue numbers of each well after reading florescence in wells to quantify cell viability. Averaged numbers of cell viability are shown below for each column indicating no detectable toxicity for either compound.

FIG. 10C shows relative HCV FRET rates for the individual wells. Compound-1453, the BVDV specific compound (Columns 1-5, A-H), is shown to have no effect on the HCV rates in each well while the HCV specific compound, (Columns 6-10, A-H) is shown to greatly decrease the NS3 protease activity.

FIG. 10D shows the relative BVDV luciferase activity following FRET assay. Compound-1453 is shown to greatly decrease the BVDV specific luciferase activity while the HCV inhibitor has no effect on the luciferase activity contained in the BVDV replicon cells.

These results indicate that assays of the present invention are useful for determining the toxicity of a test compound towards a cell, the ability of that test compound to inhibit HCV activity and the selectivity of that test compound for HCV over other viruses. This inhibitor assay titration format shown in this Example can be modified to single point for HTS and used to readily find non-toxic compounds specific for HCV inhibition.

### DISCUSSION

Assays of the present invention may be conducted in a 96-well format, as demonstrated by the dose response curve generated by IFN-α and yields results comparable to qRT-PCR, and are amenable to an even greater degree of miniaturization, such as a 384 or smaller based cell culture assay.

As illustrated in FIGS. 7A and 7B, assays of the present invention are capable of measuring toxicity associated with a test compound as well as inhibitory activity associated with the test compound in the same well, thereby providing a method to prioritize compounds according to their inhibitory profile versus HCV as well as according to their toxicity profile. The variation associated with such assays is also statistically acceptable, as illustrated in FIG. 8. The cytotoxicity assay reagents, such as Alamar Blue, are desirably easily removed and are not deleterious to the cells.

Moreover, the use of cells containing the BVDV replicon which incorporate a reporter gene, allow for an assay step in which the specificity of a test compound for HCV may be determined. Accordingly, assays of the present invention include at least three assay steps which are desirably performed in a single well of a tissue culture plate: (1) the toxicity of a test compound may be determined by Alamar blue conversion; (2) the ability of a test compound to inhibit HCV activity, as measured by NS3 FRET peptide cleavage may be determined; and (3) the ability of a test compound to inhibit BVDV activity, as measured by reporter expression, may be determined. By measuring the inhibition of BVDV activity, the selectivity of the test compound for HCV over BVDV may be determined. These three measurements determined which compounds were non-toxic, inhibited HCV and were specific for HCV.

Moreover, assays of the present invention have distinct advantages when compared to qRT-PCR or other methods in that assays of the present invention may take place in-situ in a detergent based crude cell lysate, which requires no further preparation prior to performing the assays. Assays of the present invention do not involve numerous manipulations to add or subtract reagents after addition of test compounds, and are desirably based on a viral protein which is required by the HCV replicon for replication. The FRET protease substrate peptide, which is resistant to cleavage by endogenous Huh-7 cellular proteases over the assay time period, is efficiently recognized by the replicon-based NS3 enzyme. Given that the original purpose of the substrate was to monitor the in-vitro cleavage (Taliani, supra) of this substrate by purified rather than crude enzyme, it is known that the substrate can still be cleaved by the many different genotypes of HCV NS3, thereby providing greater utility.

While the invention has been described in connection with specific embodiments therefore, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. All references cited herein are expressly incorporated in their entirety.

1. A cell-based assay for identifying a compound that inhibits HCV RNA replication and has specificity for HCV, comprising the steps of:
(a) providing a first cell which expresses at least one enzyme associated with HCV RNA replication;
(b) providing a second cell comprising a viral replicon which is not a HCV replicon;
(c) contacting said first cell and said second cell with a test compound;
(d) determining whether said test compound inhibits HCV RNA replication in said first cell;
(e) determining whether said test compound is cytotoxic to said first cell; and
(f) determining whether said test compound inhibits the activity of said viral replicon which is not a HCV replicon in said second cell.

2. The cell-based assay of claim 1, wherein said first cell comprises a HCV replicon.

3. The cell-based assay of claim 1, wherein said viral replicon which is not a HCV replicon is a BVDV replicon.

4. The cell-based assay of claim 2, wherein said HCV replicon comprises a polynucleotide having the nucleic acid sequence set forth in SEQ ID NO:1.

5. The cell-based assay of claim 2, wherein said HCV replicon encodes a polypeptide having the amino acid sequence set forth in SEQ ID NO:2.

6. The cell-based assay of claim 2, wherein said HCV replicon comprises the molecular construct set forth in FIG. 1.

7. The cell-based assay of claim 1, wherein said first cell which expresses at least one enzyme associated with HCV RNA replication is a cell having ATCC Accession No. PTA-4583.

8. The cell-based assay of claim 1, where in said viral replicon which is not a HCV replicon incorporates a reporter gene.

9. The cell-based assay of claim 8, wherein said reporter gene is luciferase.

10. The cell-based assay of claim 8, wherein said step (f) of determining whether said test compound is specific for said first cell is accomplished by measuring the activity of said reporter gene.

11. The cell-based assay of claim 1, wherein said enzyme associated with HCV RNA replication is a protease.

12. The cell-based assay of claim 11, wherein said protease is a serine protease

13. The cell-based assay of claim 12, wherein said serine protease is NS3 protease.

14. The cell-based assay of claim 11, wherein said enzyme is NS4A.

15. The cell-based assay of claim 1, wherein said step of determining whether said test compound inhibits HCV RNA replication is conducted by contacting said first cell with a fluorescence substrate.

16. The cell-based assay of claim 15, wherein said fluorescence substrate is a FRET peptide.

17. The cell-based assay of claim 1, wherein said step of determining whether said test compound is cytotoxic to said cell is conducted by contacting said first cell with an Alamar Blue solution.

18. The cell-based assay of claim 1, wherein said cell-based assay is performed in a high-throughput manner.

19. A compound identified by the cell-based assay of claim 1.

20. A pharmaceutical composition comprising a compound of claim 19.

21. A method for treating hepatitis-C, comprising the step of administering to a mammalian species in need thereof a therapeutically effective amount of a compound of claim 19.

22. A cell-based assay for identifying a compound that inhibits HCV RNA replication and has specificity for HCV, comprising the steps of:
(a) providing a first cell which expresses at least one enzyme associated with HCV RNA replication;
(b) providing a second cell which comprises a viral replicon which is not a HCV replicon and which includes a reporter gene;
(c) contacting said first cell and said second cell with a test compound;
(d) contacting said first cell with a compound which permits the determination of whether said test compound inhibits HCV RNA replication;
(e) contacting said first cell with an indicator solution which permits the determination of whether said test compound is cytotoxic to said cell; and
(f) measuring the expression of said reporter gene to determine if said test compound is specific for affecting the activity of said enzyme associated with HCV RNA replication.

23. The cell-based assay of claim 22, wherein said compound which permits the determination of whether said test compound inhibits HCV RNA replication is a FRET peptide.

24. The cell-based assay of claim 22, wherein said indicator solution which permits the determination of whether said test compound is cytotoxic to said cell is an Alamar Blue solution.

25. The cell-based assay of claim 22, wherein said reporter gene is luciferase.

26. The cell-based assay of claim 22, wherein steps (a), (b), (c), (d), (e) and (f) are conducted in a single well.

27. A cell-based assay for identifying a compound that inhibits HCV RNA replication and has specificity for HCV, comprising the steps of:
(a) providing a first cell which expresses at least one enzyme associated with HCV RNA replication, said first cell comprising a HCV replicon;
(b) providing a second cell comprising a BVDV replicon which incorporates a reporter gene;
(c) contacting said first cell and said second cell with a test compound;
(d) contacting said first cell with a FRET peptide for determining whether said test compound inhibits HCV RNA replication;
(e) contacting said first cell with an indicator solution for determining whether said test compound is cytotoxic to said cell; and
(f) measuring the expression of said reporter gene.

28. The cell-based assay of claim 27, wherein said indicator solution is an Alamar Blue solution.

29. The cell-based assay of claim 27, wherein said HCV replicon comprises a polynucleotide having the nucleic acid sequence set forth in SEQ ID NO:1.

30. The cell-based assay of claim 27, wherein said HCV replicon encodes a polypeptide having the amino acid sequence set forth in SEQ ID NO:2.

31. The cell-based assay of claim 27, wherein said HCV replicon comprises the molecular construct set forth in FIG. 1.

32. The cell-based assay of claim 27, wherein said cell which expresses at least one enzyme associated with HCV RNA replication is a cell having ATCC Accession No. PTA-4583.

33. A compound identified by the cell-based assay of claim 27.

35. A pharmaceutical composition comprising a compound of claim 33.

36. A method for treating hepatitis-C which comprises administering to a mammalian species in need thereof a therapeutically effective amount of a compound of claim 33.

37. A cell-based assay for identifying a compound that inhibits HCV RNA replication and has specificity for HCV, comprising the steps of:
(a) providing a first cell having ATCC Accession No. PTA-4583, said first cell expressing at least one enzyme associated with HCV RNA replication;
(b) providing a second cell comprising a BVDV replicon which incorporates a reporter gene;
(c) contacting said first cell and said second cell with a test compound;
(d) contacting said first cell with a FRET peptide for determining whether said test compound inhibits HCV RNA replication;
(e) contacting said first cell with an indicator solution for determining whether said test compound is cytotoxic to said cell; and
(f) measuring the expression of said reporter gene.

38. The cell-based assay of claim 3, wherein said BVDV replicon comprises a polynucleotide having the nucleic acid sequence shown in FIG. 9.

39. The cell-based assay of claim 22, wherein said viral replicon which is not a HCV replicon comprises a polynucleotide having the nucleic acid sequence shown in FIG. 9.

40. The cell-based assay of claim 27, wherein said BVDV replicon comprises a polynucleotide having the nucleic acid sequence shown in FIG. 9.
